(19) United States
(12) Patent Application Publication
(10) Pub. No.: US 2022/0168172 A1
(43) Pub. Date: Jun. 2, 2022

(54) Wearable decentralized apparatus for optimal remote ischemic preconditioning

(71) Applicant: Fazal RAHEMAN, Nagpur (AE)

(72) Inventor: Fazal RAHEMAN, Nagpur (AE)

(21) Appl. No.: 17/442,152

(22) PCT Filed: Jun. 27, 2019
(86) PCT No.: PCT/IB2019/055436

§ 371 Date: Sep. 23, 2021

(30) Foreign Application Priority Data
Mar. 25, 2019 (CN) 2019102292473

(51) Int. Cl. A61H 9/00; G16H 20/30; G16H 40/67; H04W 12/06

(52) U.S. Cl. CPC A61H 9/0092; G16H 20/30; G06Q 20/389; H04W 12/06; G16H 40/67

(57) ABSTRACT

The invention discloses an improvised decentralized network of wearable optimal remote ischemic preconditioning (ORIP) apparatus for optimally harnessing the innate power of homeostasis for inducing system-wide preconditioning that protect all vital organs from subsequent physiological or pathological insults. More particularly it discloses a wearable medical device that not only maximizes multi-organ protection in minimum dosing time, but also enhances apparatus portability, operability, networkability, security, dosing safety and compliance, user confidentiality and incentivized peer participation with social media engagement for content creation, curation, sharing and monetization.

### FIELD OF THE INVENTION

The present invention generally relates to optimal remote ischemic preconditioning (ORIP) methods of transiently and repeatedly auto inflating pneumatic cuffs for inducing limb ischemia distal to its site of occlusion and preconditioning the entire human body to subsequent physiological and pathological insults. More particularly it relates to wearable medical device that not only maximizes multi-organ protection in minimum dosing time, but also enhances apparatus portability, operability, networkability, security, dosing safety and compliance, user confidentiality and incentivized peer participation with social media engagement for content creation, curation, sharing and monetization.

### BACKGROUND

This invention improvises on U.S. Pat. No. 8,911,469 disclosing an apparatus and method of optimal remote ischemic preconditioning (ORIP) that harnesses the innate power of homeostasis by using a pair of programmable pneumatic pneumatic cuffs that inflate and deflate alternately for a pre-specified time to induce repetitive intermittent transient ischemia in limbs to induce optimal limb ischemia sufficient for inducing system-wide preconditioning that protects all vital organs from physiological or pathological insults. The improvements are primarily in the following areas:
1. Network security and privacy.
2. Enhanced dosing safety & compliance
3. Enhanced device portability & wearability
4. Incentivizing and motivating user participation via relevant content distribution and influence monetization.

There is a need to address aforementioned issues for enhancing the user experience and for improving performance of the apparatus. As reasons therefore present invention discloses systems, apparatus and methods to create, verify, and maintain a cloud-based decentralized network infrastructure of highly portable, secure and hack-proof ORIP devices that make the devices private, safe, compliant and social media friendly. Such decentralized network infrastructure generally relates to autonomous, permissionless, distributed databases more particularly to blockchains. Blockchain are replicated ledgers of transactions between peers that save their data in sequential data files at each of the peer nodes. Such peer-to-peer transactions or smart contracts are verified in blockchain by having multiple computer systems hash the data describing a smart contract transaction and when a majority of computer systems compute the same hash, the transaction is considered verified and added to the replicated ledgers. In simpler terms the present invention discloses a computer-implemented system, apparatus and method of utilizing an autonomous decentralized system of blockchain's distributed ledger infrastructure and self-executing smart contracts for securely authenticating the device user, maintaining user's confidentially, ensuring the safety and compliance of user's dosing regimen, and at the same time facilitating participating peers' social media engagement for content creation, curation, sharing and monetizing their influence. Directed Acyclic Graph (DAG) is another alternate technology to implement a decentralized network of the present invention. It is to these ends that the present invention has been developed.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. If the specification states a component or feature “may”, “can”, “could”, or “might” be included or have a characteristic, that particular component or feature is not required to be included or have the characteristic.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

### SUMMARY

It would be an improvement to provide a novel apparatus and decentralized network infrastructure of highly portable, wearable, secure and hack-proof ORIP devices that make the induction of optimal remote ischemic preconditioning therapy in patient's portable, confidential, safe, compliant and social media friendly. It would also be an improvement to deploy blockchain for decentralizing the network. It would also be an improvement to authenticate, authorize, verify and immutably record each ORIP dosing session as a self-executing smart contract on a public or private ledger. As reasons therefore this invention discloses a home based self-operating wearable health and wellness device that ensures confidentially, safety and compliance of user's dosing regimen, and at the same time facilitates participating peers' social media engagement for their health related content creation, sharing, curation, and monetizing their content and their influence.

Accordingly, this invention discloses a computer implemented, decentralized Internet-connected wearable apparatus for improving general health and wellness of users by means of self-administering optimal remote ischemic preconditioning (ORIP) treatment safely and securely for preventing or attenuating all kinds of pathological or physiological injuries to organs. It would therefore be an object to provide a system wherein an autonomous decentralized network secures, anonymously monitors, personalizes and incentivizes peer participation and abuse-free compliance to ORIP (Optimal Remote Ischemic Preconditioning) treatment regimen. It is also an object to non-invasively render maximal systemic protection against diverse acute or chronic physiological or pathological insults to organs and morbidities resulting thereof. It would therefore be an object to provide a wearable apparatus comprising of a pair of wireless wearable devices with built-in, auto-inflatable, pneumatic cuffs that induce transient, repetitive and alternating ischemia and reperfusion of at least a pair of remote organs including a first limb and a second contralateral limb, resulting in release of ischemia triggered cellular modulators (ITCMs) or redox molecules, which circulate throughout the body improving physiological functions of malfunctioning or underperforming vital organs or tissues, and increasing their threshold to various physiological or pathological insults resulting in prevention or attenuation of tissue injury and consequential alleviation of consequent pathological conditions thereof.

It is an object to provide a method of activities authentication and self-executing smart contract that defines treatment regimen verified by consensus of participating peers and immutably maintained in a database of distributed ledger amongst the participating nodes. It is therefore a consequent object to deploy either a blockchain protocol or a Directed Acyclic Graph (DAG) protocol for decentralizing the network of ORIP devices by means of wirelessly connecting each pair of ORIP devices to a handheld mobile device that functions as a node in the decentralized network. It is further object to seamlessly operate the paired auto-inflatable pneumatic cuffs via the mobile device, by authenticating the user, the paired ORIP devices, the treatment regimen and the validity of treatment session to exclude unauthorized usage securely by means of a single touch. It is also a further object to monitor the treatment anonymously to ensure privacy and compliance via creation of immutable distributed ledger of ORIP administered dosing sessions administered making treatment abuse impossible. It is yet another object of the invention to provide the use of traditional centralized database infrastructure where decentralized blockchain infrastructure cannot be implemented.

It is also further object of the invention to incentivize peer participation by means of analyzing and delivering to users prognostic parameters of improved health effects of ORIP treatment, sharing and delivering monetizable user-generated content or sponsored content to users during the ORIP dosing sessions to subsidize the ORIP treatment costs, and providing microblogging interface for creating revenue generating opportunities for participating peers. It is therefore a further object to tokenize peer participation with zero transaction fee blockchain that produces monitory rewards proportional to the content creation or content curation activity the peers conduct, transaction blocks they produce, and stakes they hold within the platform's token economy. It would therefore be a consequent object to provide a mobile GUI that encourages the users to share their ORIP experience with the network of peers, and reward such user-generated content with tokenized author and curation rewards mined as a result of each blockchained transaction, resulting in not only building their peer-rated influence but monetizing such influence.

It is yet another object to remove all controls from the paired devices, except the power button, and provide a single virtual button within the mobile application GUI to operate the paired ORIP devices in tandem. It is further object of the invention to limit ORIP treatment sessions to once a day in chronic ambulatory use cases and not more than twice in 24 hours in acute use cases such as in pre-procedure treatment of interventional procedures that cause substantial tissue injuries that include but not limited to angioplasty, bypass surgery, or high does radiation such as in angiography or radiation therapies.

It is still further object of the invention to optimally induce the production of ischemia triggered cellular modulators (ITCMs) or redox molecules that include a plethora of tissue protective cellular modulators such as stress proteins, cytokines, redox signaling molecules, antioxidants, all of which collectively optimize body's homeostasis in maintaining body in a healthy state and decelerate aging.

It is the ultimate object of the present invention to prevent, alleviate or attenuate pathophysiological conditions such as acute tissue injury episodes such as myocardial infarction or cerebral stroke, and many acute and chronic consequences of endothelial dysfunction, the hallmark of human diseases, which include a plethora of consequent morbidities such as hypertension, atherosclerosis, sleep apnea, Raynaud's disease, hypertension, portal hypertension, diabetes, renal failure, pregnancy/pre-eclampsia, diabetic foot, stroke, dementia, macular degeneration, Alzheimer's disease, periodontal disease, heart attack, heart failure, angina, pulmonary hypertension, erectile dysfunction, peripheral arterial disease (PAD), cancer, tumor growth, metastasis, venous thrombosis, and severe viral infectious diseases.

The foregoing discussion summarizes some of the more pertinent objects of the present invention. These objects should be construed to be merely illustrative of some of the more prominent features and applications of the invention. Applying or modifying the disclosed invention in a different manner can attain many other beneficial results as will be described in detail herein. Accordingly, referring to the following drawings may have a complete understanding of the invention and its preferred embodiments

Various objects, features, aspects and advantages of the inventive subject matter will become more apparent from the following detailed description of preferred embodiments, along with the accompanying drawing figures in which like numerals represent like components.

### BRIEF DESCRIPTION OF DRAWINGS

The accompanying drawings are included to provide a further understanding of the present disclosure, and are incorporated in and constitute a part of this specification.

The drawings illustrate exemplary embodiments of the present disclosure and, together with the description, serve to explain the principles of the present disclosure. The diagrams are for illustration only, which thus is not a limitation of the present disclosure, and wherein:

FIG. 1 is a block diagram illustrating a novel embodiment of the wearable ORIP apparatus.

FIG. 2 is a block diagram illustrating the decentralized network of a novel embodiment of the invention.

FIG. 3 is a block diagram illustrating the network architecture of the incentivized and decentralized ORIP therapy system.

FIG. 4 is a block diagram illustrating the range of morbidities impacted by ORIP's improved endothelial function.

FIG. 5 is a flow-chart describing a remote ORIP procedure in a distributed ledge in a decentralized environment.

### DETAILED DESCRIPTION OF THE INVENTION

In the following description, numerous specific details are set forth in order to provide a thorough understanding of embodiments of the present invention. It will be apparent to one skilled in the art that embodiments of the present invention may be practiced without some of these specific details.

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware and/or by human operators.

A Blockchain is a decentralized distributed database that maintains a continuously growing list of data records hardened against tampering and revision. It consists of data structure blocks, which hold exclusively data in earlier blockchain implementations, and both data and programs in some of the more recent implementations, with each block holding batches of individual transactions and the results of any blockchain executables. Although originally a backbone of cryptocurrencies, blockchain protocols can be a boon to any P2P infrastructure where peers can seamlessly self-execute smart contracts securely without having to disclose their identities. Directed Acyclic Graph (DAG) is an alternate decentralizing technology that is faster and lighter than blockchain.

A blockchain can typically be understood to be a distributed database that maintains a continuously growing list of records called blocks. Each block contains a link to a previous block generated using a hash of the previous block, and often includes mechanisms for protection from tampering and revision. The blockchain is distributed so that copies are replicated among participating nodes in the system. As transactions are added the copies are extended and the longest chains are trusted that follows a rule and provides according proof of work or stake. Nodes may be any of a variety of hardware devices, such as, but not limited to, servers, workstations, desktop computers, mobile devices, tablets and/or wearable devices, configured to participate in the decentralized digital system as discussed further below.

In a number of embodiments, the blockchain ledger is stored and verified in a decentralized fashion, i.e. replicated to different entities (that may be referred to as nodes) that are separately able to verify past transactions. In further embodiments, the ownership of a creation as well as license can be proven using cryptographic principles. In many embodiments, the chaining of transactions, cryptographic verification and the hash challenge utilizes a blockchain system based on principles and/or mechanisms similar to those in the art of cryptocurrency. Techniques for managing blockchains in the context of currency are described in “Bitcoin: A Peer-to-Peer Electronic Cash System” by Satoshi Nakamoto, published in 2008, the disclosure of which relevant to blockchain management is hereby incorporated by reference in its entirety.

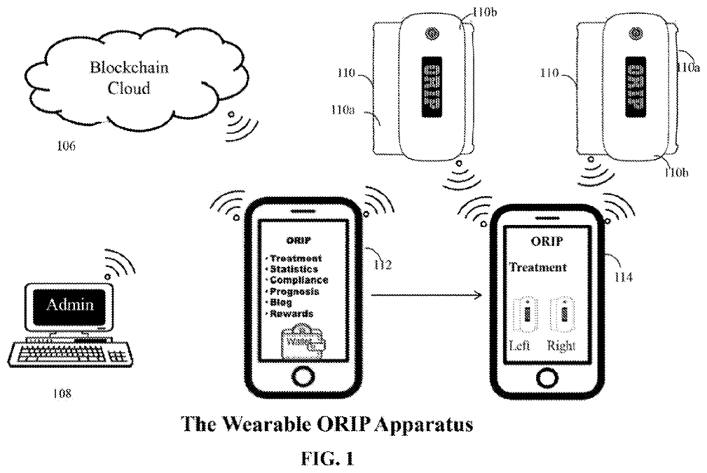

Exemplary embodiments will now be described more fully hereinafter with reference to the accompanying drawings (FIGS. 1-5), in which exemplary embodiments are shown. These exemplary embodiments are provided only for illustrative purposes and so that this disclosure will be thorough and complete and will fully convey the scope of the invention to those of ordinary skill in the art. The invention disclosed may, however, be embodied in many different forms and should not be construed as limited to the embodiments set forth herein. Various modifications will be readily apparent to persons skilled in the art.

The core feature of the instant invention is decentralized, seamless, autonomous, safe, secure and incentivized network of apparatus comprising of a pair of compact ORIP (Optimal Remote Ischemic Precondition) devices 110, each device comprising of a tubeless pneumatic cuff 110a that directly attaches to an assembly 110b of air pump, pressure transducer, and microprocessor. The automatic flexible inflatable and deflatable cuff occludes the brachial artery when inflated beyond the subject's systolic blood pressure thereby inducing a zone of ischemia in the limb tissue distal to the pneumatic cuff.

In an exemplary embodiment, the automatic flexible inflatable and deflatable pneumatic cuffs that induce transient, repetitive and alternating ischemia and reperfusion of at least a pair of remote organs including a first limb and a second contralateral limb, resulting in release of ischemia triggered cellular modulators (ITCMs) or redox molecules, which by circulating throughout the body improve the physiological functions of malfunctioning or underperforming vital organs or tissues, and further increasing their threshold to various physiological or pathological insults, resulting in prevention or attenuation of tissue injury and consequential alleviation of consequent pathological conditions.

The ischemia triggered cellular modulators (ITCMs) or redox molecules include a plethora of tissue protective cellular modulators that include but not limited to stress proteins, cytokines, redox signaling molecules, antioxidants, all of which collectively optimize body's homeostasis in maintaining body in a healthy state and decelerate aging.

In an exemplary embodiment, every ORIP treatment comprising a pair of auto-inflatable pneumatic cuffs, begin with attaching a first cuff to the first limb and a second cuff to the second limb, inducing transient, repetitive and alternating ischemia and reperfusion cycles numbering between 2 and 4 cycles, each cycle being not less than 2 minutes and not more than 5 minutes long, which is achieved by means of raising cuff pressure to not less than 5 mm and not more than 50 mm beyond systolic blood pressure measured at the beginning of the first ischemic cycle.

In an exemplary embodiment, the treatment sessions are prescribed once a day in chronic ambulatory use cases and not more than twice in 24 hours in acute use cases such as pre-procedure treatment of interventional procedures that cause substantial tissue injuries that include but not limited to angioplasty, bypass surgery, or high dose radiation therapies such as in angiography or radiation therapies.

In an exemplary embodiment, every treatment session begins and ends with blood pressure measurements in each limb for measuring and recording immediate effects of ORIP treatment on cardiovascular system in general and endothelial function in particular.

In an exemplary embodiment, a method of authentication and self-executable smart contract that defines treatment regimen verified by consensus of participating peers and immutably maintained in a database of distributed smart ledger amongst the participating nodes.

In an exemplary embodiment, the decentralized network either utilizes a blockchain protocol or a Directed Acyclic Graph (DAG) protocol for decentralizing the network of ORIP devices by means of wirelessly connecting each pair of ORIP devices to a handheld mobile device, which functions as a network node with a mobile application.

In an exemplary embodiment, the handheld mobile device and/or any other computing device exclusively operate the previously paired auto-inflatable pneumatic cuffs, thus delivering to the user the ORIP treatment dose securely with a single touch.

In an exemplary embodiment, the blockchain environment authenticates the user, the devices, the treatment regimen and the validity of treatment session to exclude unauthorized usage or procedure.

In an exemplary embodiment, the peers monitor the treatment anonymously to ensure privacy and compliance via creation of immutable distributed ledger of ORIP administered dosing sessions administered making treatment abuse impossible. The ledgers are stored as per the decentralized architecture of the blockchain technology.

In an exemplary embodiment, the novel blockchain based ORIP system incentivizes the peer participation by means of analyzing and delivering to users prognostic parameters of improved health effects of ORIP treatment.

In an exemplary embodiment, the system further share and deliver the monetizable user-generated content or sponsored content to users during the ORIP dosing sessions to further incentivize and subsidize the ORIP treatment costs.

In an exemplary embodiment, the system further provides a micro-blogging and/or a social network interface for generating revenue by creating content sharing opportunities for participating peers.

As illustrated in FIG. 1, the infrastructure for implementing the wearable ORIP apparatus in accordance with an exemplary embodiment of the present disclosure. It would be appreciated that aspects of the present disclosure can be applied to a variety of network architectures, all of which are well within the scope of the present disclosure.

In an exemplary embodiment, network architecture of the present disclosure can include a wearable ORIP apparatus having/connected with one or more Internet of Things (IoT) devices in a blockchain environment. The wearable ORIP apparatus includes a left and a right ORIP wearable device 110, having 110a being the pneumatic cuff ORIP while 110b being the assembly of air pump, pressure transducer, and microprocessor. The pneumatic cuffs may be wrapped around the user's limb such as wrist or arm or any other limb of the body. Both wearable devices 110 are wirelessly connected to the handheld mobile device and/or any other computing device.

As used herein, the IoT devices can be a device that includes sensing and/or control functionality as well as a WiFi™ transceiver radio or interface, a Bluetooth™ transceiver radio or interface, a Zigbee™ transceiver radio or interface, an Ultra-Wideband (UWB) transceiver radio or interface, a WiFi-Direct transceiver radio or interface, a Bluetooth™ Low Energy (BLE) transceiver radio or interface, and/or any other wireless network transceiver radio or interface that allows the IoT device to communicate with a wide area network and with one or more other devices. In some embodiments, an IoT device does not include a cellular network transceiver radio or interface, and thus may not be configured to directly communicate with a cellular network. In some embodiments, an IoT device may include a cellular transceiver radio, and may be configured to communicate with a cellular network using the cellular network transceiver radio.

In an exemplary embodiment, the mobile device and/or any other computing device is connected to the administrator/admin 108, and connected with wearable ORIP devices 110 in a blockchain network environment.

The mobile device and/or other computing device having a web interface while processing computer readable instructions to administer ORIP on the patient. The interface may be implemented as a standalone application or a web based application running on the smartphone and/or any other computing device having similar capabilities. The interface give the user or the practitioner the ability to administer the ORIP treatment along with options to connect to the peers for sharing the user-generated content on one or more social media platforms for building and monetizing influence. As the method is implemented in the blockchain environment or a decentralized environment, the privacy and other information will not directly link to the patient, medical practitioner and to the peers. Along with that, the option of using the blog for real-time posting is also available.

In an exemplary embodiment, the rewards in the form of crypto-currencies or tokens are provided to the participating peers. The rewards in the form of crypto-currencies or tokens may also be stored or collected in a wallet within the ORIP mobile application. Also each user activity is recorded as transactions or smart contracts and stored in the decentralized environment of blockchain.

In an exemplary embodiment, each of the paired devices is operated in tandem exclusively by means of a single virtual button within the mobile application user interface. This provides a simple mechanism to administer the ORIP using web interface of the mobile application. Also, there are no device-resident controls except the power button on the wireless wearable device. This ensures safety and prevents abuse.

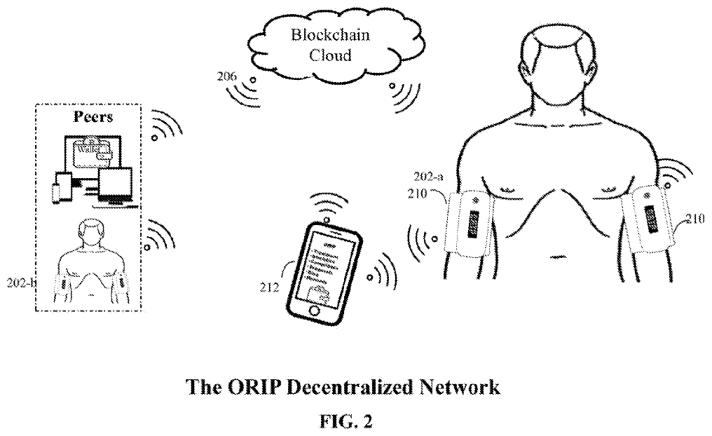

FIG. 2 is a block diagram illustrating the ORIP decentralized network, in accordance with an exemplary embodiment of the present disclosure.

In an exemplary embodiment, the ORIP treatment is self-administered by wearing a pair of ORIP devices on each of the user's (202-a), upper arms, wrists or any other body limb by wrapping the pneumatic cuffs around in a similar manner as used in a conventional blood pressure measuring devices but operated exclusively via an application running on mobile or any other computing device. As specified earlier each ORIP therapy session is authorized and authenticated using blockchain 206 validation process and immutably recorded in the blockchain database. Likewise other peers (202-b) of the user also operate the ORIP device and connect to the blockchain cloud 206 for authentication and validation of their ORIP treatment session. The peers can also interact with each other by securely sharing their experience with the community via a blogging interface present within the ORIP mobile application. Such content sharing is incentivized in terms of rewards generated in the form of tokens mined via the blockchain's transaction/smart contract validation protocol discussed in more detail subsequently. Thus the web-interface and/or the application running on mobile or any other computing device not only operates the ORIP apparatus for securely and safely administering the ORIP treatment but it connects users anonymously to the decentralized blockchain cloud for socializing, sharing content, building influence and getting rewarded for their content.

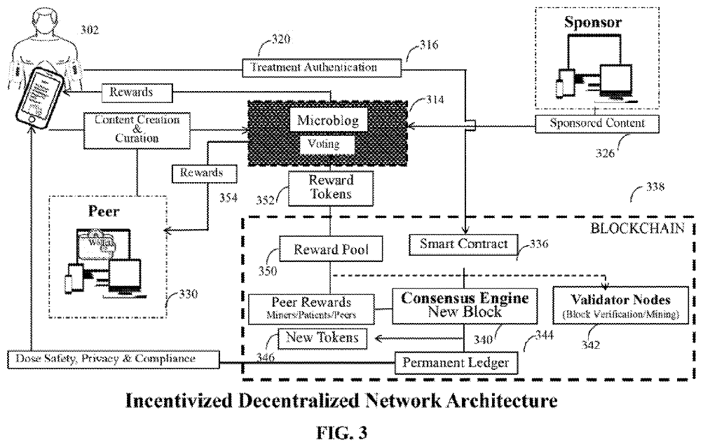

FIG. 3 is a block diagram illustrating the network architecture of the incentivized and decentralized ORIP therapy system, in accordance with the exemplary embodiment of the present disclosure.

The platform uses a low or ultra-low latency blockchain network 300 not only to decentralize the network of peers, but to maintain the immutable records of all transactions with highest possible security, privacy, anonymity, and in the process generating or mining new tokens (funds) using a transaction validation consensus protocol that includes but not limited to proof-of-work, proof-of-stake, delegated proof-of-stake or their variants thereof.

The user 302 wears a pair of compact ORIP devices 302a, having tubeless pneumatic cuffs attached to the user on left and right limbs. The paired ORIP devices are wirelessly connected to a handheld mobile device or any other mobile computing device 302b, which has an application platform running that authenticates 304 and initiates the ORIP treatment session by pressing a single virtual button on the application interface. Each treatment session is a smart contract 306 that gets recorded by the blockchain's consensus engine 308 as a new block validated by block validator or miner nodes 310 and stored in blockchain' s database of distributed ledger 312.

As an incentive to the block validators or miners 310, the consensus engine produces new tokens 314 as validation rewards 316 to the validators/miners 310. The distributed ledger 312 stores and also feeds the user application interface 302b with the information regarding the dose safety, privacy & compliance 318. The peer rewards may be distributed amongst the peer nodes as rewards tokens either to the block validators 312 directly, or pooled in as reward pool 320 to be distributed as reward tokens 322 or tokenized votes for content creation and curation activities 324 of the peers on the platform's blog or microblog 326. The blog votes represent the quantum of tokenized influence of participating peers, which can be converted into spendable currency.

Along with content created and curated by participating peers, sponsored content 328 can also be posted to the microblogging website. The sponsored content also generates revenue, which may be shared with the participating peers. The sponsored content can be provided by any individual, agency or corporate. Based on the quality of the content posted and curated, the rewards 330a & 330b are distributed to the user who creates it and to the peers 332 who curate it.

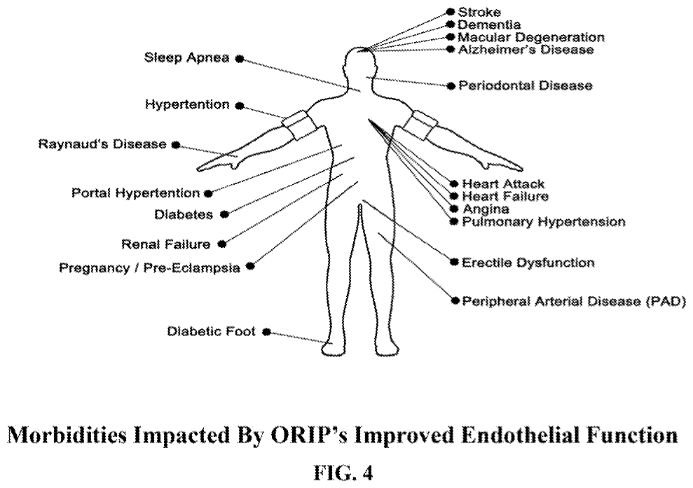

FIG. 4 is a block diagram illustrating the range of morbidities impacted by ORIP's improved endothelial function in accordance with an exemplary embodiment of the present invention. In an exemplary embodiment, the range of morbidities impacted by ORIP's improved endothelial functions are described but not limited to such as Stroke, Dementia, Macular Degeneration, Alzheimer, periodontal disease, Sleep Apnea, Hypertention, Raynaud Disease, Portal Hypertention, Diabetes, Renal Failure, Pregnancy/Pre-Eclampsia, Heart Attack, Heart failure, Angina, Pulmonary Hypertention, Erectile Dysfunction, Peripheral Arterial Disease (PAD) and Diabetic Foot.

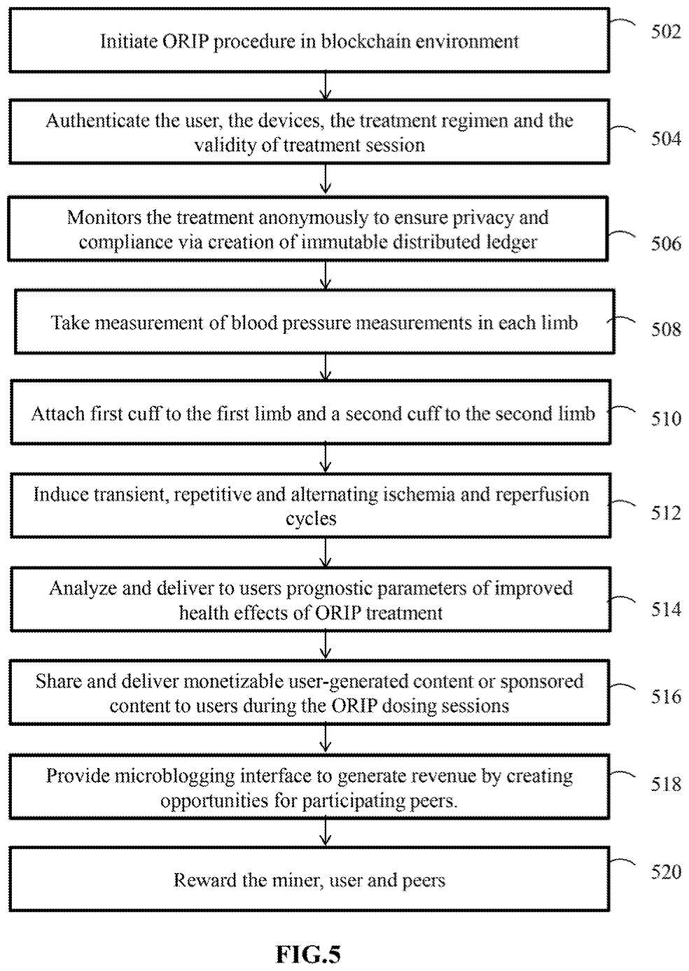

FIG. 5 is a flow-chart of steps involved in administrating ORIP procedure in accordance with an exemplary embodiment of the present disclosure, which begins with user attaching first pneumatic cuff to first limb and second pneumatic cuff attached to the second limb, and powering on the paired devices to wirelessly connect the paired device to user's mobile device 502. At block 504 the ORIP procedure is initiated by clicking on the “Start” button on the web application interface on user's mobile device.

At block 506 the mobile application authenticates the user, the devices, the treatment regimen and the validity of treatment session, and immutably records the events on blockchain. The ORIP session starts by taking blood pressure measurements in the first or both limbs for determining the systolic pressure for inducing ischemia distal to the cuff 508.

At block 510 the paired devices induce transient, repetitive, alternating ischemia and reperfusion cycles in each limb by holding cuff pressure above systolic for a period of not less than 2 minutes and not more than 5 minutes. The apparatus operates until at least 2 to 4 cycles each of alternating ischemia and reperfusion are administered. Such ORIP treatment session is monitored by blockchain, and the treatment administered safely and anonymously to ensure privacy and compliance, and prevent abuse by creation of immutable distributed ledger 512.

At block 514, the system analyzes and delivers the prognostic parameters of improved health effects of ORIP treatment to the user. This motivates the user for increasing treatment compliance, and also promotes community engagement if the user shares the positive results with peers. The microblogging interface built within the mobile application 516 not only allows secure blogging and sharing where they can share the experiences and advice/feedback, but also creates revenue-generating opportunities by monetizing the content. At block 518 the system shares delivers monetizable user-generated content or sponsored content to users during the ORIP dosing sessions. This results in subsidizing the ORIP treatment costs and may even make it free.

At block 520 the platform's tokenomics generates rewards in the form of tokens or cryptocurrency, and delivers the cryptocurrency tokens to the wallets of the miners, content creators and peers.

Embodiments of the present invention may be provided as a computer program product, which may include a machine-readable storage medium tangibly embodying thereon instructions, which may be used to program a computer (or other electronic devices) to perform a process. Various methods described herein may be practiced by combining one or more machine-readable storage media containing the code according to the present invention with appropriate standard computer hardware to execute the code contained therein. An apparatus for practicing various embodiments of the present invention may involve one or more computers (or one or more processors within a single computer) and storage systems containing or having network access to computer program(s) coded in accordance with various methods described herein, and the method steps of the invention could be accomplished by modules, routines, subroutines, or subparts of a computer program product.

The general principles defined herein may be applied to other embodiments and applications without departing from the spirit and scope of the invention. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

Thus, for example, it will be appreciated by those of ordinary skill in the art that the diagrams, schematics, illustrations, and the like represent conceptual views or processes illustrating systems and methods embodying this invention. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this invention. Those of ordinary skill in the art further understand that the exemplary hardware, software, processes, methods, and/or operating systems described herein are for illustrative purposes and, thus, are not intended to be limited to any particular named element.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illustrate the invention and does not pose a limitation on the scope of the invention otherwise claimed. No language in the specification should be construed as indicating any non-claimed element essential to the practice of the invention.

In an exemplary embodiment, the ORIP device may include at least one processor, an input/output (I/O) interface, and a memory. At least one processor may be implemented as one or more microprocessors, microcomputers, microcontrollers, digital signal processors, central processing units, state machines, logic circuitries, and/or any devices that manipulate signals based on operational instructions. Among other capabilities, at least one processor is configured to fetch and execute computer-readable instructions stored in the memory.

Although the proposed system has been elaborated as above to include all the main modules, it is completely possible that actual implementations may include only a part of the proposed modules or a combination of those or a division of those into sub-modules in various combinations across multiple devices that can be operatively coupled with each other, including in the cloud. Further the modules can be configured in any sequence to achieve objectives elaborated. Also, it can be appreciated that proposed system can be configured in a computing device or across a plurality of computing devices operatively connected with each other, wherein the computing devices can be any of a computer, a laptop, a smartphone, an Internet enabled mobile device and the like. All such modifications and embodiments are completely within the scope of the present disclosure.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the spirit of the appended claims.

What is claimed is:

1. A safe, secure, private, portable, wearable, networkable, abuse-free computer-implemented system, apparatus and platform for utilizing an autonomous decentralized network to secure, anonymously monitor, personalize and incentivize peer participation and compliance to ORIP (Optimal Remote Ischemic Preconditioning) treatment regimen that non-invasively renders maximal systemic protection against diverse acute or chronic physiological or pathological insults to organs and morbidities resulting thereof, by means of a pair of wireless wearable devices with built-in, auto-inflatable, pneumatic pneumatic cuffs that induce transient, repetitive and alternating ischemia and reperfusion of at least a pair of remote organs including a first limb and a second contralateral limb, resulting in release of ischemia triggered cellular modulators (ITCMs) or redox molecules, which circulate throughout the body improving physiological functions of malfunctioning or underperforming vital organs or tissues, and increasing their threshold to various physiological or pathological insults resulting in prevention or attenuation of tissue injury and consequential alleviation of consequent pathological conditions thereof.

2. A computer-implemented system, apparatus and platform of claim 1 wherein the autonomous decentralized network utilizes either a blockchain protocol or a Directed Acyclic Graph (DAG) protocol for decentralizing the network of ORIP devices by means of wirelessly connecting each pair of ORIP devices to a handheld mobile device, which functions as a network node with a mobile application that:
exclusively operates the paired auto-inflatable pneumatic pneumatic cuffs seamlessly, delivering to the user the ORIP treatment dose securely with a single touch,
authenticates the user, the devices, the treatment regimen and the validity of treatment session to exclude unauthorized usage,
monitors the treatment anonymously to ensure privacy and compliance via creation of immutable distributed ledger of ORIP administered dosing sessions administered making treatment abuse impossible,
incentivizes peer participation by means of:
analyzing and delivering to users prognostic parameters of improved health effects of ORIP treatment,
sharing and delivering monetizable user-generated content or sponsored content to users during the ORIP dosing sessions to subsidize the ORIP treatment costs,
providing microblogging interface for creating revenue generating opportunities for participating peers.

3. The computer-implemented platform of claim 1 wherein the decentralized network of peers is blockchained for security, privacy, anonymity, using low-latency, zero transaction fee blockchain protocol that is tokenized for incentivizing peer participation by means of producing monitory rewards proportional to the content creation or content curation activity the peers conduct, transaction blocks they produce, and stakes they hold within the platform's token economy.

4. The wireless wearable devices of claim 1 wherein each of the paired devices is operated in tandem exclusively by means of a single virtual button within the mobile application user interface, and as such has no device-resident controls except the power button.

5. The ORIP treatment regimen of claim 1 wherein each treatment session with a pair of auto-inflatable pneumatic pneumatic cuffs, comprise of attaching a first cuff to the first limb and a second cuff to the second limb, inducing transient, repetitive and alternating ischemia and reperfusion cycles numbering between 2 and 4 cycles, each cycle being not less than 2 minutes and not more than 5 minutes long, which is achieved by means of raising cuff pressure to not less than 5 mm and not more than 50 mm beyond systolic blood pressure measured at the beginning of the first ischemic cycle.

6. The ORIP treatment regimen of claim 1 wherein the treatment sessions are prescribed once a day in chronic ambulatory use cases and not more than twice in 24 hours in acute use cases such as in pre-procedure treatment of interventional procedures that cause substantial tissue injuries that include but not limited to angioplasty, bypass surgery, or high does radiation such as in angiography or radiation therapies.

7. The ischemia triggered cellular modulators (ITCMs) or redox molecules of claim 1 wherein ITCMs include a plethora of tissue protective cellular modulators that include but not limited to stress proteins, cytokines, redox signaling molecules, antioxidants, all of which collectively optimize body's homeostasis in maintaining body in a healthy state and decelerate aging.

8. The apparatus and system of claim 1 wherein the pathophysiological conditions prevented or alleviated include but not limited to an acute episode of tissue injury such as myocardial infarction or cerebral stroke.

9. The apparatus and system of claim 1 wherein the physiological and pathological insults prevented or attenuated include but not limited to acute and chronic consequences of endothelial dysfunction, the hallmark of human diseases, which include a plethora of consequent morbidities that include but not limited to hypertension, atherosclerosis, sleep apnea, Raynaud's disease, hypertension, portal hypertension, diabetes, renal failure, pregnancy/pre-eclampsia, diabetic foot, stroke, dementia, macular degeneration, Alzheimer's disease, periodontal disease, heart attack, heart failure, angina, pulmonary hypertension, erectile dysfunction, peripheral arterial disease (PAD), cancer, tumor growth, metastasis, venous thrombosis, and severe viral infectious diseases.

10. The platform of claim 1 wherein peer participation is incentivized by creating a user interface that encourages the users to share their ORIP experience with the network of peers and by rewarding such user-generated content with tokenized author and curation rewards mined as a result of each blockchained transaction, resulting in not only building their peer-rated influence but monetizing such influence.

11. The ORIP treatment regimen of claim 1 wherein each treatment session begins and ends with blood pressure measurements in each limb for measuring and recording immediate effects of ORIP treatment on cardiovascular system in general and endothelial function in particular.
